(19) United States
(12) Patent Application Publication
(10) Pub. No.: US 2021/0020191 A1
(43) Pub. Date: Jan. 21, 2021

(54) METHODS AND SYSTEMS FOR VOICE PROFILING AS A SERVICE

(71) Applicant: DeepConvo Inc., Pittsburgh, PA (US)

(72) Inventors: Satya Venneti, Pittsburgh, PA (US); Mir Mohammed Daanish Ali Khan, Pittsburgh, PA (US); Rajat Kulshreshtha, Pittsburgh, PA (US); Prakhar Pradeep Naval, Pittsburgh, PA (US); Rita Singh, Pittsburgh, PA (US)

(21) Appl. No.: 16/931,429

(22) Filed: Jul. 16, 2020

Related U.S. Application Data
(60) Provisional application No. 62/875,918, filed on Jul. 18, 2019.

(51) Int. Cl. G10L 25/78; G10L 25/63; G10L 21/0208; G06F 3/16; G06F 3/01; G06K 9/62; G06N 20/00

(52) U.S. Cl. CPC G10L 25/78; G10L 25/63; G10L 21/0208; G06F 3/16; G06F 3/011; G06F 2203/011; G06K 9/6232; G06K 9/6256; G06N 20/00; G10L 2025/783; G06K 9/6261

(57) ABSTRACT

Methods, systems and computer products as described herein are directed to a Voice Profiler. According to various embodiments, the Voice Profiler receives audio data that includes a representation of a human voice. The Voice Profiler extracts voiced segments, non-voiced segments and respiratory event segments from the input audio data. The Voice Profiler predicts a physical state of the speaker of the human voice based on respective attributes of the extracted segments. According to various embodiments, the Voice Profiler predicts the lung function of the speaker based on input audio data.

### CROSS-REFERENCE TO RELATED APPLICATIONS

This application claims the benefit of U.S. Provisional Application No. 62/875,918, filed Jul. 18, 2019, which is hereby incorporated by reference in its entirety.

### FIELD OF INVENTION

The present technology pertains to voice profiling. In particular, but not by way of limitation, the present technology provides systems and methods of voice profiling as a service.

### BACKGROUND

The approaches described in this section could be pursued, but are not necessarily approaches that have previously been conceived or pursued. Therefore, unless otherwise indicated, it should not be assumed that any of the approaches described in this section qualify as prior art merely by virtue of their inclusion in this section.

In the field of computer science, artificial intelligence (“AI”) networks, such as neural networks and deep learning networks, are increasingly being employed to solve a variety of tasks and challenging problems, often with great success. Such AI networks can consist of computational graphs with nodes representing computational operations (“ops”), and connections in between those ops. Each op computes something on input data in order to produce output data. Within AI networks there are fairly predefined ops, and there can be, e.g., hundreds or thousands of these ops. Such ops can represent such computational tasks as matrix multiplication and convolution, often using many pieces of input data from within the network. An AI network can be thought of at one level as a list of ops, or operations, that get executed in a particular order to produce an output.

### SUMMARY

By making voice profiling widely available as a service, the present technology enables a wide range of applications capable of addressing unmet needs in the areas of medical diagnostics, drug detection, forensics, security, and the like. While the commercial opportunities in each of these areas are unique to the use cases in those spaces, there is generally a common need to find more cost-effective and scalable methods of characterizing people and their current mental and physical attributes.

For many reasons, voice data is an ideal source of this information. Voice data can be collected passively (e.g., as a byproduct of regular human interaction) using widespread, existing infrastructure (e.g., mobile phones). The present technology offers widespread, affordable medical screening and real-time awareness of physical and mental states of people by profiling voice as a service. The present technology provides access to the corresponding benefits to all corners of the world and dramatically lowers costs everywhere.

Methods, systems and computer products as described herein are directed to a Voice Profiler. According to various embodiments, the Voice Profiler receives audio data that includes a representation of a human voice. The audio data represents a recording of a session during which a speaker of the human voice responded to one or more prompts. The Voice Profiler extracts voiced segments, non-voiced segments and respiratory event segments from the input audio data. The Voice Profiler predicts a physical state of the speaker of the human voice based on respective attributes of the extracted segments. According to various embodiments, the Voice Profiler predicts the lung function of the speaker based on the audio data.

Various end users may use different types of computer devices in varied settings. As such, implementing a technique that accounts for a default or generic amount of background noise will not effectively address the differing levels of background noise actually present in recordings from sessions of different individual speakers. To ensure that the background noise in each audio input file is properly removed (“de-noised”) on a per-session basis, the Voice Profiler calibrates the background noise for each individual speaker's audio input file. Stated differently, the Voice Profiler performs first and second level segmentation of the input audio file to apply a background noise calibration (i.e. background noise profile, background profile) specific to an end user's session. During first level segmentation, the background noise calibration is used to segment out prompt responses in the input audio file in a binary fashion between positive segments and background +negative segments. The first level segmentation achieves denoising and segmentation simultaneously. During second level segmentation, the background noise calibration and segments (i.e. first level output) are further segmented in a binary fashion into background noise segments and inhale/exhale segments.

It is understood that privacy and security functionalities may be included in all embodiments described herein in order to anonymize personal identification data and health data of end-users in order to protect the privacy of end-users. For example, for an implementation of the Voice Profiler service via a cloud-based platform, data sent to and from Voice Profiler modules may be anonymized and/or encrypted prior to receipt and transmission. In addition, any personal identification data and health data stored via the cloud-based platform may be further scrubbed of unique identifiers that a 3^{rd}-party may use to identify an end-user.

Various embodiments described herein are directed to voice profiling as a service, comprising: (a) receiving an audio file of a human voice from a human or a representation of the human voice; (b) receiving desired attribute predictions of the human; and (c) providing a plurality of attribute predictions of the human with accompanying confidence values based on the desired attribute predictions of the human using pre-trained classifiers. In various embodiments, representation of the human voice is an encrypted binary file from which the audio file of the human voice from the human cannot be reconstructed.

According to various embodiments, the input audio data may include audio portions that correspond to vocal actions performed by the speaker in response to instructions provided by one or more prompts. Various machine learning segmentation models may be applied to the audio portions to extract various types of segments isolated from background noise present in the input audio data. During a first level of machine learning segmentation, the Voice Profiler extracts de-noised voiced segments, de-noised forced exhale segments, pause segments and inhale-background segments. The pause segments include inhale and exhale audio with background noise. The inhale-background segments include inhale audio with background noise. The Voice Profiler extracts features from the respective segments extracted during the first pass to calculate various types of metrics, such as word rate and average pauses per minute. It is understood that extracted segments are homogeneous regions in the input audio data.

During a second level of machine learning segmentation, the Voice Profiler sends the pause segments and the inhale-background segments generated from first level segmentation as input for the various machine learning segmentation models. The Voice Profiler receives, from the second level segmentation, de-noised inhale segments and de-noised exhale segments. After the second level, the Voice Profiler inputs the de-noised inhales segments, the de-noised exhale segments, the de-noised forced exhale segments (from the first level) and the de-noised voiced segments (from the first level) as input into one or more machine learning classifier models. The Voice Profiler receives classified segments that include speech segments, coughing segments and wheezing segments. The Voice Profiler extracts features from the classified segments to calculate various types of predicted metrics for the physical state of the speaker, such as lung functioning metrics for forced vital capacity (FVC) and forced expiratory volume (FEV).

According to various embodiments, the Voice Profiler converts input audio to a spectrogram representation of the input data and analyzes various regions in the spectrogram representation according to respective differences in frequency signal intensities. The Voice Profiler detects a region of the spectrogram representation that exceeds an intensity threshold and extracts a voiced segment from the input audio that maps to the detected region of the input spectrogram. The Voice Profiler predicts the physical state of the speaker based in part on the extracted voiced segment.

According to various embodiments, the Voice Profiler may predict the physical state of the speaker whereby the physical state may be related to a degree of lung conditioning for athletic performance, the presence of a mental health condition (such as stress, anxiety, depression), asthma, chronic obstructive pulmonary disease (COPD).

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1 is a diagram illustrating an exemplary environment in which some embodiments may operate.

FIG. 2A is a flowchart illustrating exemplary methods that may be performed in some embodiments.

FIG. 2B is a diagram illustrating an exemplary environment in which some embodiments may operate.

FIG. 2C is a diagram illustrating an exemplary environment in which some embodiments may operate.

FIG. 3A is a flowchart illustrating exemplary methods that may be performed in some embodiments.

FIG. 3B is a flowchart illustrating exemplary methods that may be performed in some embodiments.

FIG. 4 is a diagram illustrating an exemplary environment in which some embodiments may operate.

FIG. 5 is a diagram illustrating an exemplary environment in which some embodiments may operate.

FIG. 6 is a diagram illustrating an exemplary environment in which some embodiments may operate.

FIG. 7 is a diagram illustrating an exemplary environment in which some embodiments may operate.

FIGS. 8A and 8B provide a listing of calculated and predicted physical state metrics according to some embodiments.

FIG. 9 is a diagram illustrating an exemplary computer that may perform processing in some embodiments.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

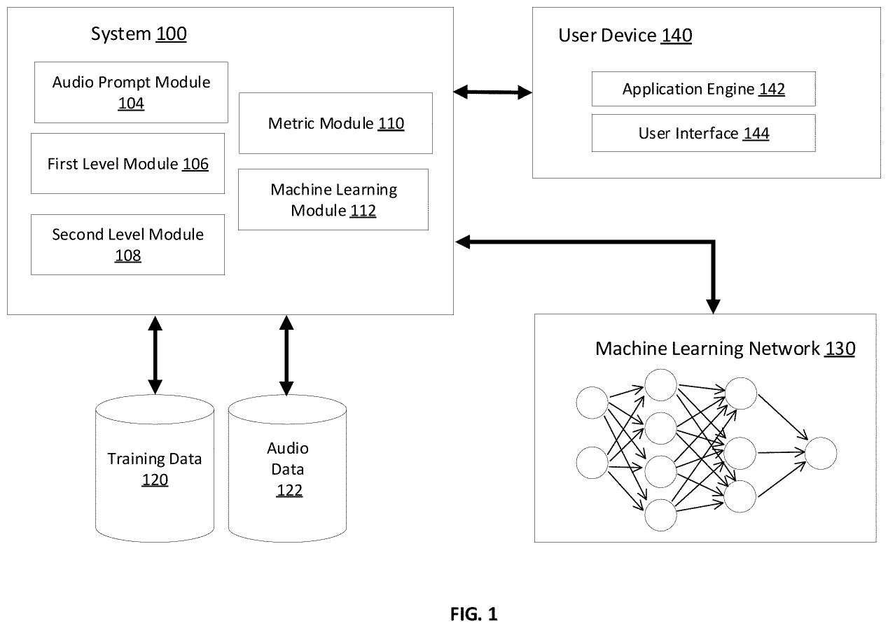

FIG. 1 is a diagram illustrating an exemplary environment in which some embodiments may operate. FIG. 1 illustrates a block diagram of an example system 100 that includes an audio prompt module 104, a first level module 106, a second level module 108, a metric module 110, a training module 112 and a machine learning module 114. The system 100 may communicate with a user device 140 to display output, via a user interface 144 generated by an application engine 142. The machine learning network 130 and the databases 120, 122 may further be components of the system 100 as well.

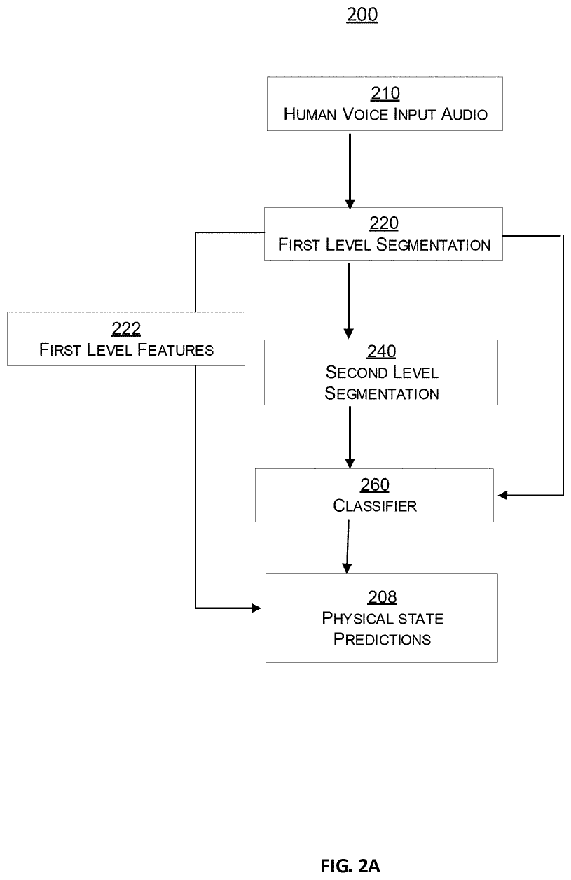

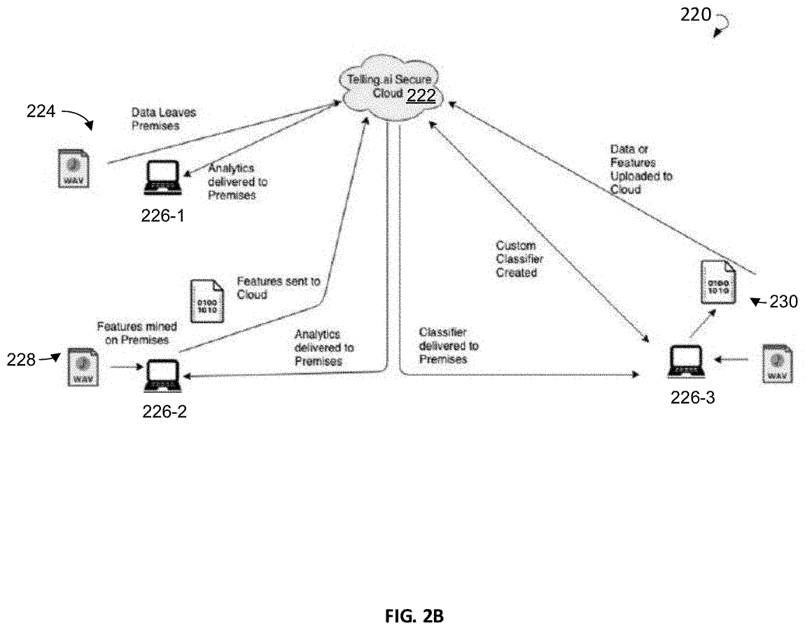

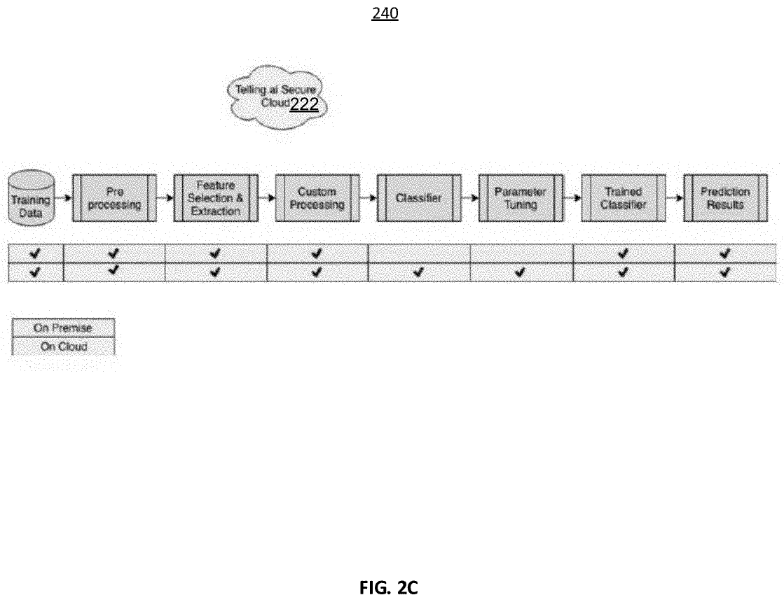

The audio prompt module 104 of the system 100 may perform functionality for identifying input audio generated in response to prompts as illustrated in FIGS. 2A, 2C, 3A, 4 and 5.

The first level module 106 of the system 100 may perform functionality for first level segmentation and first level feature extraction as illustrated in FIGS. 2A, 2C, 3A, 3B, 4 and 5.

The second level module 108 of the system 100 may perform functionality for second level segmentation, classification and second level feature extraction as illustrated in FIGS. 2A, 2C, 3A, 4, 6 and 7.

The metric module 110 of the system 100 may perform functionality as illustrated in FIGS. 2A, 2C, 3A, 4, 5, 6, 7, 8A and 8B.

The machine learning module 112 of the system 100 may perform for accessing and implementing the machine learning network 130 during first and second level segmentation.

While the databases 120, 122 are displayed separately, the databases and information maintained in a database may be combined together or further separated in a manner the promotes retrieval and storage efficiency and/or data security.

As shown in flowchart 200 of FIG. 2A, the Voice Profiler receives human voice audio 210 and uses the human voice audio 210 as input for first level segmentation 220. First level segmentation 220 generates various types of segments extracted from the input audio 210 via machine learning segmentation. The Voice Profiler extracts features 220 from the first level segments and computes metrics 208 based on the first level features. The Voice Profiler uses as input for second level segmentation 240 the one or more extracted segments from first level segmentation 220. Second level segmentation 240 includes further machine learning segmentation to generate as output second level segments. The second level segments are input into a classifier model 260. The classifier model 260 returns classified segments. The Voice Profiler extracts features from the classified segments and uses the second level features to compute predictive metrics 208 regarding the physical state of the speaker represented in the human voice audio 210. It is understood that the extracted segments from the first level and the second level are homogeneous regions in the input audio data.

As shown in FIG. 2B, a Voice Profiler service implemented via a cloud-based platform 222 may receive and provide data according to various embodiments. According to a first embodiment 224, audio data may be received at the cloud-based platform 222. First and second level segmentation, feature extraction, metric calculation and prediction may occur at the cloud-based platform 222. Analytics resulting from metric calculation and prediction may be securely sent back to a computer system 226-1.

According to a second embodiment 228, one of more Voice Profiler modules may be executed on a computer system 226-2 for performing first and second level segmentation and feature extraction at the computer system 226-2 in response to receiving input audio data. Extracted features may be securely sent to the cloud-based platform 222. Voice Profiler modules executed via the cloud-based platform 222 generate metric calculations and predictions based on the received extracted features. Analytics resulting from metric calculations and predictions may be securely sent back to the computer system 226-2.

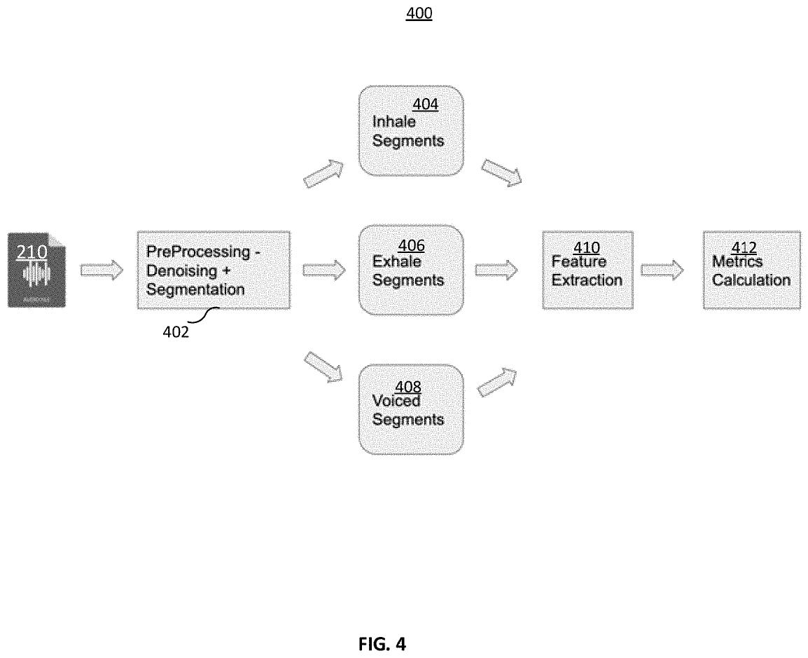

According to a third embodiment 230, a second level classifier model may be sent from the cloud-based platform 222 to a computer system 226-3. Voice Profiler modules may be executed on the computer system 226-3 for performing first and second level segmentation and feature extraction at the computer system 226-2 in response to receiving input audio data. As shown in a diagram 240 of FIG. 2C, various stages of the Voice Profiler service may be executed at the cloud-based platform 222 and various stages may also be executed on a computer system(s) remotely located from the cloud-based platform 222 (i.e. “on premise”). It is understood that the pre-processing, feature selection & extraction, custom processing, classifier stages as illustrated in FIG. 2C correspond to the embodiments described in connection with FIGS. 4, 5, 6 and 7.

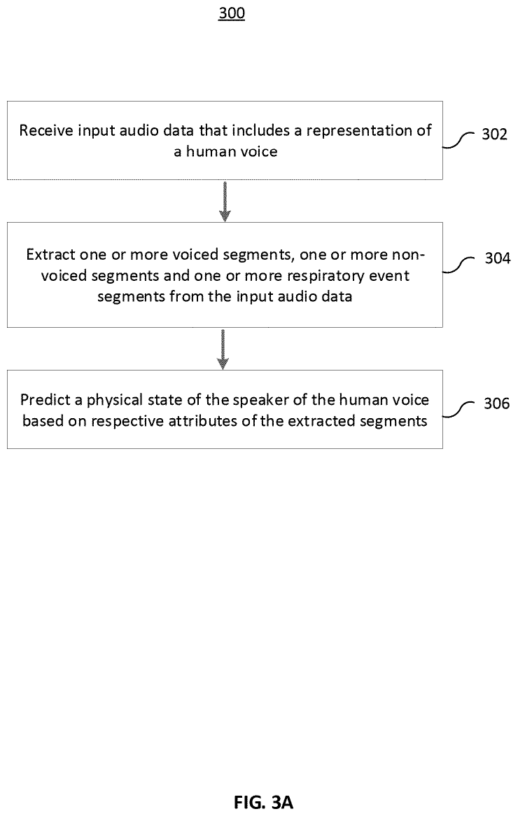

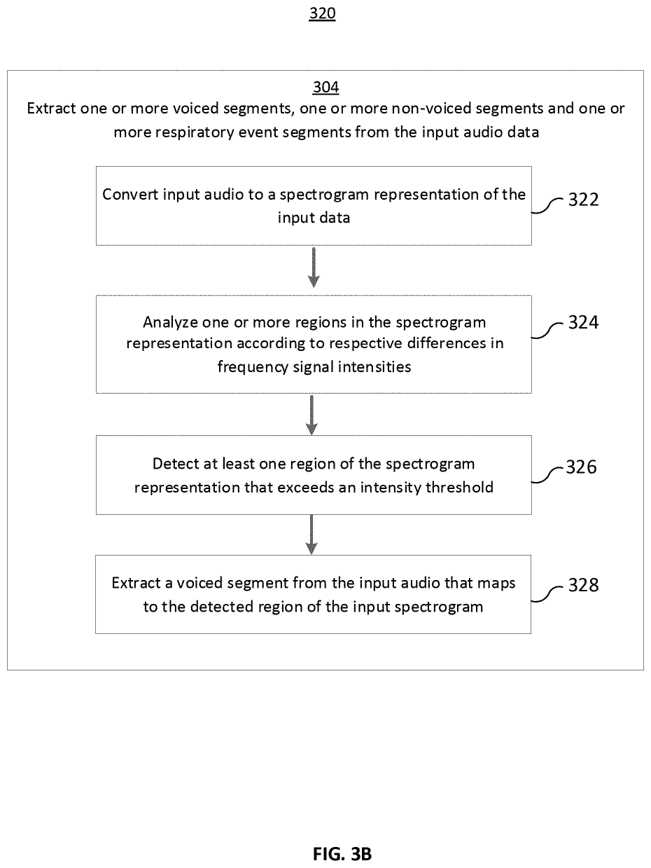

As shown in flowchart 300 of FIG. 3, the Voice Profiler receives input audio data that includes a representation of a human voice (Act 302) and extracts one or more voiced segments, one or more non-voiced segments and one or more respiratory event segments from the input audio data (Act 304). Respective audio portions in the input audio data correspond to vocal actions performed by the speaker in response to instructions provided by one or more prompts. For example, a prompt may be: a prompt to remain silent, one or more types of speech prompts and one or more types of non-speech prompts. Respective types of respiratory events include respective inhale occurrences and respective exhale occurrences during the vocal actions performed by the speaker.

Segment extraction includes first level and second level segmentation implemented by the Voice Profiler. During first level segmentation, the Voice Profiler extracts certain types of segments from the input audio. Isolation of background noise in the input audio happens in parallel with first level segment extraction. For example, the Voice Profiler determines a per session background profile based on the background noise present in the input calibration audio data (such as a .wav file or an mp3 file). The Voice Profiler applies one or more machine learning segmentation models to the input audio data and input based on the background noise calibration (i.e. background profile). The Voice Profiler receives, from the first level machine learning segmentation models, various types of extracted segments isolated from the background noise calibration, such as: de-noised voiced segments, de-noised respiratory event inhale segments and de-noised respiratory event exhale segments. For the second level segmentation, the Voice Profiler implements various machine learning segmentation models. Input for the second level segmentation is based on one or more first level extracted segments and the background noise calibration. The Voice Profiler uses the second level output extracted segments as input to a machine learning classifier model. The classifier model returns speech segments, cough segments, wheezing segments, throat clearing segments, crackle segments and/or stridor sound segments.

The Voice Profiler predicts a physical state of the speaker of the human voice based on respective attributes of the extracted segments (Act 306). The respective attributes correspond to features extracted from segments produced during first and second segmentation. After first level segmentation, the Voice Profiler extracts features from the first level extracted segments in order to compute various metrics. After the second level segmentation, the Voice Profiler extracts features from the second level segments returned by the classifier model in order to compute predictive metrics.

As shown in flowchart 320 of FIG. 3B, during first level segment extraction, the Voice Profiler converts input audio to a spectrogram representation of the input data (Act 322) A spectrogram representation comprises data based on the spectrum of frequencies of the audio signal(s) in the input audio. The Voice Profiler analyzes regions in the spectrogram representation according to respective differences in frequency signal intensities (Act 324). The Voice Profiler detects a region of the spectrogram representation that exceeds an intensity threshold (Act 326) and extracts a voiced segment from the input audio that maps to the detected region of the input spectrogram (Act 328). The Voice Profiler predicts the physical state of the speaker based on, in part, on the extracted voiced segment.

It is understood that some of the acts of the exemplary flowcharts 300 and 320 may be performed in different orders or in parallel. Also, one or more of the acts in the exemplary flowcharts 300 and 320 may occur in two or more computers, for example if the method is performed in a networked environment. Various acts may be optional. Some acts may occur on local computer with other acts occur on a remote computer.

As shown in FIG. 4, the Voice Profiler sends the input audio 210 through a first level segmentation pipeline 402 which includes various machine learning models, parallel denoising, morphological and smoothing operations implemented by the Voice Profiler. Output from the first level segmentation pipeline 402 includes various types of segments extracted from the input audio 210. The first level extracted segments include inhale segments 404, exhale segments 406 and/or voiced segments 408. The Voice Profiler extracts features from the one or more of the output segments 404, 406, 408 and computes various first level metrics 412.

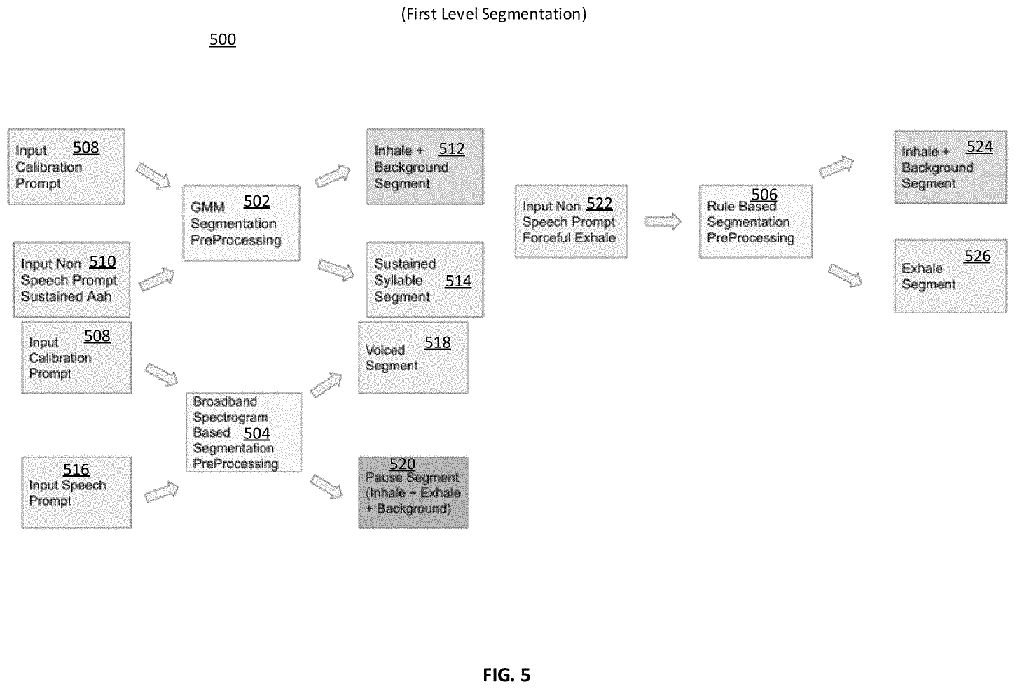

As shown in FIG. 5, during the first level segmentation, the Voice Profiler receives input audio portions and uses a background noise calibration to segment out a “de-noised” positive segment and a background segment. Such extracted “de-noised” positive segments may be sustained syllable segment, a voiced segment and a forced exhale segment. According to various embodiments, first level segmentation may utilize a variety of machine learning models to which audio portions are input and the generated output may be a “de-noised” positive segment extracted from the input audio and a background segment extracted from the input audio such that it is isolated from the extracted “de-noised” positive segment.

First Level: De-Noised Sustained Syllable Segments

According to an embodiment, the Voice Profiler identifies audio portions 508 that correspond to the speaker refraining from speaking in response to a prompt to remain silent. Such audio portions 508 are used to calibrate the background noise present in the audio data files pertaining to the session. The audio portions 508 thereby represent the calibrated background noise that is to be isolated from input audio portions. The Voice Profiler also identifies audio portions 510 that correspond to the speaker performing a sustained syllable sound in response to a prompt to make a sustained syllable sound. The audio portions 508, 510 are input into a Gaussian Mixture machine learning segmentation model 502 (“GMM model”) implemented by the Voice Profiler in order to extract one or more “de-noised” sustained syllable segments 514 apart from one or more isolated inhale +background segments 512.

According to various embodiments, the GMM model 502 uses input background noise 508 to learn the background noise in the input audio 210 as a result of treating the background noise as a mixture of Gaussian distributions. For example, each audio frame that is part of the input background noise 508 is represented as being sampled from a respective Gaussian distribution. To compute a probability of any audio frame being generated by the background noise mixture, the Voice Profiler extracts spectral features from the background noise audio and sustained “aah” audio 510 from sustained syllable prompts. The extracted calibration spectral features are then fit into a GMM model 502. A posterior likelihood of GMM on each frame of the sustained aah spectral features is computed and a smoothing process is applied to each frame's posterior throughout the input audio file 210. The Voice Profiler defines a posterior threshold that identifies all respective segments of audio frames that fall below threshold as candidate segments that represent phonation. The Voice Profiler selects a phonation candidate region that represents a highest total energy (i.e. duration & energy) and determines an energy threshold based on an average of audio frame energies in the selected phonation candidate region. The Voice Profiler fine-tunes selected phonation candidate region starting point based on the energy threshold. The Voice Profiler extracts one or more segments from audio frames that occur after the starting point which have a low posterior likelihood as segments that are representative of non-background noise and thereby include a sustained syllable segment 514. It is understood that the extracted inhale +background segment 512 includes audio of an inhalation performed by the speaker for the sustained syllable as well as audio that is representative of the calibrated background noise 508 occurring while the speaker performs the sustained syllable.

First Level: De-Noised Voiced Segments & Pause Segments

The Voice Profiler identifies voiced input audio that corresponds to vocal actions performed in response to speech prompts and converts the voiced input audio into a spectrogram representation of the voiced input data. The Voice Profiler analyzes regions in the voiced input spectrogram representation according to respective differences in frequency signal intensities and detects a region of the voiced input spectrogram representation that exceeds an intensity threshold. The Voice Profiler extracts a portion of the voiced input audio that maps to the detected region of the voiced input spectrogram and label the extracted portion as respective de-noised voiced segment.

According to various embodiments, the Voice Profiler identifies audio portions 516 that correspond to the speaker saying one or more words in response to a speaking prompt (such as a reading passage prompt or a word list prompt. The audio portions 508, 516 are input into a Broadband Spectrogram-based Segmentation machine learning model 504 in order to extract one or more “de-noised” voiced segments 518 isolated from one or more pause segments 520. The “de-noised” voiced segments 518 correspond to audio of the speaker saying one or more words in response to the speaking prompt. The one or more pause segments 520 correspond to audio of the speaker performing an inhalation and an exhalation in order to say the one or more words as well as audio that is representative of the calibrated background noise 508 occurring while the speaker says the one or more words.

According to various embodiments, the Voice Profiler implements the Broadband Spectrogram-based Segmentation machine learning model 504 in order to represent the input audio 516 according to a broadband spectrogram. The model 504 represents the spectrum of frequencies in the signal of input audio 516 (which includes the one or more words, an inhalation, an exhalation and background noise) as it varies across time. At each time step, the model 504 estimates the spectrum according to a Fast Fourier Transform in order to identify one or more broad spectral envelope peaks that correspond to speech. The model 504 defines the intensity per time step to be the sum of intensities across frequencies at that respective time step. A classification rule is applied to the summed frequency intensities per time step in order to classify an audio frame in the input audio 516 as being representative of speech segment or background noise segment.

A threshold for the classification rule is determined by an estimate of a background noise floor in the input calibrated background noise audio 508. The threshold is an intensity threshold for differentiating between speech regions and background noise regions in the spectrogram. The Voice Profiler averages the top-k values from the spectrogram to calculate an estimate of the intensities in the speech regions that represent one or more voiced segments in the input audio 516. The representation of the spectrogram is adjusted by re-setting all values quieter than (or less than) the intensity threshold (i.e. estimated speech intensity - noise floor intensity) as the noise floor.

Next, the Voice Profiler creates a classification rule to separate the foreground and background regions in the spectrogram since the mean between foreground and background intensities acts as a viable estimate of a threshold that separates speech frequencies from background noise frequencies. The Voice Profiler generates, as output, a binary classification as speech or background per time step. However, in order to account for misdetections and missed detections of speech, the Voice Profile implements morphological steps based on known aspects about the nature of pauses during speech. The Voice Profiler applies one or more morphological operations on the binary classification rule output to reduce misdetections and missed detections of speech. For example, the Voice Profiler defines a window that corresponds to an empirically determined duration of audio, which is the typical length of a pause during speech. A closing operation is used to remove very short portions of background noise detected in the middle of a speech segment. An opening operation is used to remove very short portions of speech detected in the middle of a background noise segment. A dilation operation is used to slightly broaden each speech segment and ensure no speech is left in a proceeding background noise segment.

First Level: De-Noised Forced Exhale Segments

The Voice Profiler identifies audio portions 522 that correspond to the speaker performing a forced exhale in response to a prompt for a forced exhale. The audio portions 522 are input into a Rule-based Segmentation machine learning model 506 implemented by the Voice Profiler in order to extract a “denoised” forced exhale segment 526 that corresponds to audio of the speaker performing a forced exhale in response to the force exhale prompt. An extracted inhale +background noise segment 524 is isolated from the denoised forced exhale segment 526, where the inhale +background noise segment 524 includes audio of an inhalation performed by the speaker for the forced exhale as well as audio that is representative of the calibrated background noise 508 occurring while the speaker performs the forced exhale. It is understood that a “denoised” segment is any segment that has been isolated from background noise that substantially matches the background noise calibration.

The Voice Profiler implements the model 506 to identify the intensity values in the input audio 522 over time to determine a threshold. The threshold differentiates between portions of the input audio 522 which correspond to exhalation. For example, any audio frame in the input audio 522 where the intensity is greater than the threshold is identified as representing audio of the forced exhale to be extracted as one or more “de-noised” forced exhale segments 526.

The Voice Profiler employs an additional check that a contiguous chunk of audio frames that represent the force exhalation must be greater than an empirically determined threshold. According to various embodiments, the Voice Profiler validates the audio portions identified as representing the forced exhale against ground truth data. Portions of the input audio 522 that are not identified as forced exhale segments 526 are identified as one or more inhale +background segments.

First Level: Feature Extraction & Computed Metrics

The Voice Profiler extracts one or more temporal features and/or one or more spectral features from one or more of the segmented extracted during first level segmentation. Temporal features are based on properties measurable in the time domain, such as, for example: signal energy, zero crossing rate, loudness, energy, speaking rate, articulation rate (i.e. phonation rate). Spectral domain features are based on properties measurable in the frequency domain, such as, for example: Mel-frequency cepstral coefficient, pitch and/or fundamental frequency, harmonics, spectral flux, spectral density, spectral roll-off, formant frequency, formant magnitude, formant dispersion, and/or formant bandwidth.

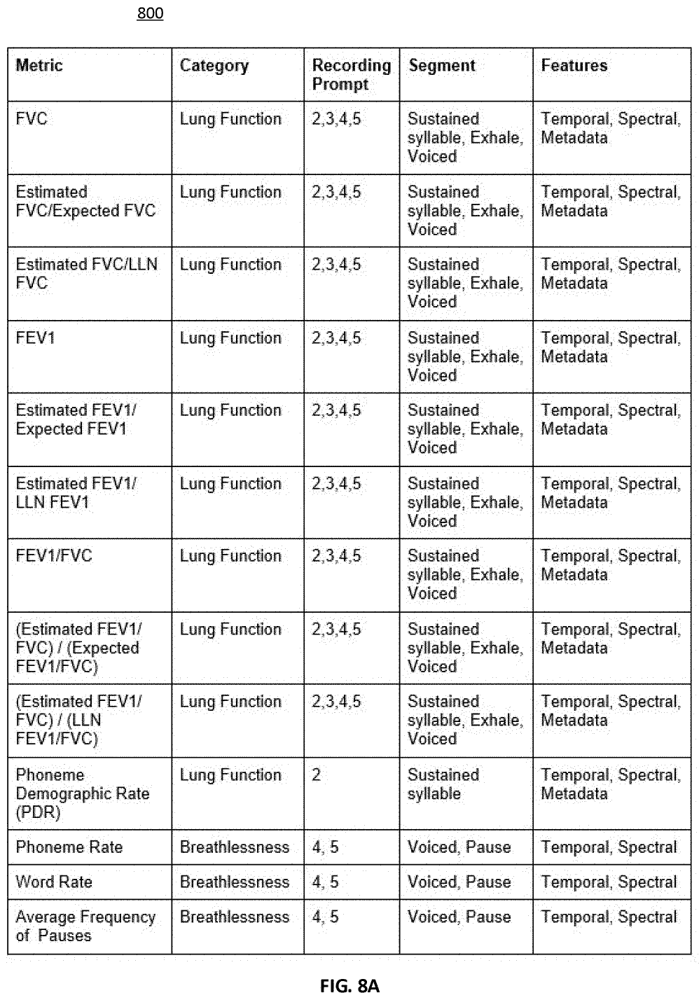

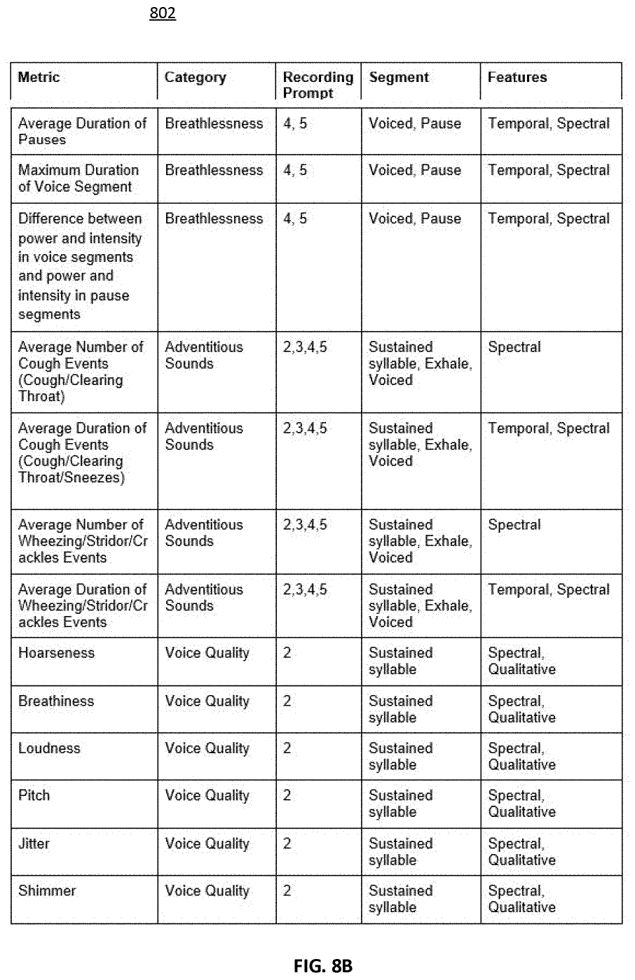

The Voice Profiler computes one or more metrics based on the first level features. Such computed metrics include, for example: Phoneme Demographic Rate, Phoneme Rate, Word Rate, Average Frequency of Pauses, Average Duration of Pauses, Maximum Duration of Pauses, Maximum Duration of Voiced Segments, and Difference between power-&-intensity in voiced segments and power-&-intensity in pause segments, as listed in the FIGS. 8A-8B.

Second Level: Rule-Based Segmentation

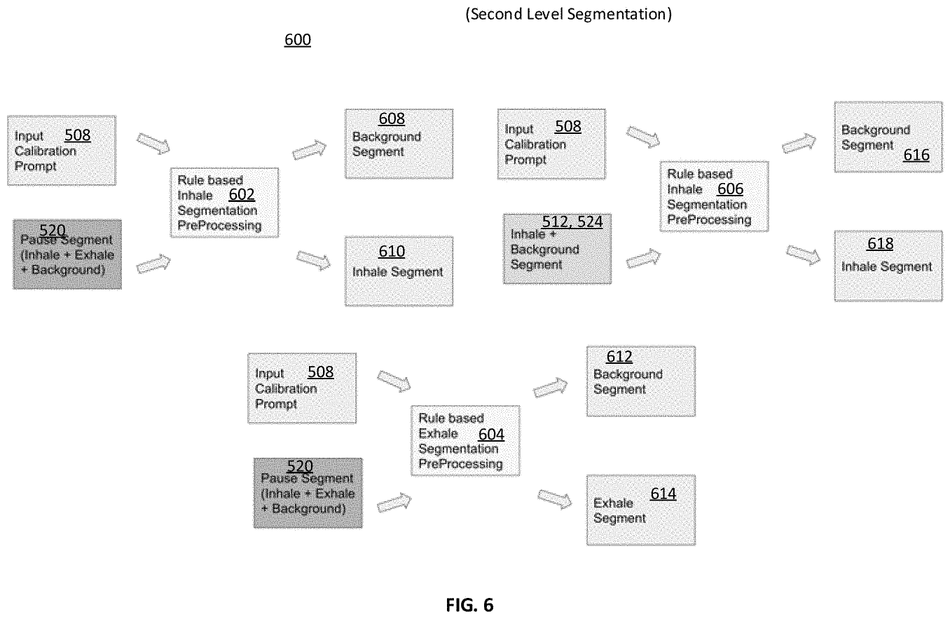

As shown in FIG. 6, second level segmentation implemented by the Voice Profiler includes utilizing segments from the first level segmentation as input to further extract positive segments isolated from background noise. For second level segmentation, the Voice Profiler implements various types of rule-based segmentation machine learning models 602, 604, 606 that operate in a similar manner as the Rule-based segmentation model 506 for the forced exhale in the first level.

The Voice Profiler implements a rule-based inhale segmentation model 602 to identify the intensity values in one or more first level pause segments 520 over time to determine a first inhale threshold. The first inhale threshold differentiates between portions of the input pause segment 520 which correspond to inhalation and other audio in the pause segment 520. For example, any audio frame in the pause segment 520 where the intensity is less than the first inhale threshold and greater than intensity of the background noise calibration 508 is identified as representing audio of an inhalation to be extracted from the input pause segment 520 and isolated from the background noise present in the pause segment 520. The rule-based inhale segmentation model 602 generates as output one or more inhale segments 610 and one or more background noise segments extracted from the input pause segment 520.

The Voice Profiler implements a rule-based exhale segmentation model 604 to identify the intensity values in one or more first level pause segments 520 over time to determine an exhale threshold. The exhale threshold differentiates between portions of the input pause segment 520 which correspond to exhalation and other audio in the pause segment 520. For example, any audio frame in the pause segment 520 where the intensity is less than the exhale threshold is identified as representing audio of an inhalation and/or background noise. Any audio frame in the pause segment 520 where the intensity is greater than the exhale threshold is identified as representing audio of an exhalation. The rule-based exhale segmentation model 604 generates as output one or more exhale segments 614 and one or more background noise segments 612 extracted from the input pause segment 520. It is understood that, in some embodiments, the pause segments 520 input into the respective models 602, 604 may be the same or may be different pause segment instances.

The Voice Profiler implements a rule-based inhale segmentation model 606 to identify the intensity values in one or more inhale +background segments 512, 514 over time to determine a second inhale threshold. The second inhale threshold differentiates between portions of the input segment 512, 524 which correspond to inhalation. For example, any audio frame in the input segment 512, 524 where the intensity is less than the second inhale threshold is identified as representing audio of background noise. Any audio frame in the input segment 512, 524 where the intensity is greater than the second inhale threshold is identified as representing audio of an inhalation. The rule-based exhale segmentation model 606 generates as output one or more inhale segments 618 and one or more background noise segments 616 extracted from the input segment 512, 524. It is understood that, in some embodiments, the pause segments 520 input into the respective models 602, 604 may be the same or may be different pause segment instances.

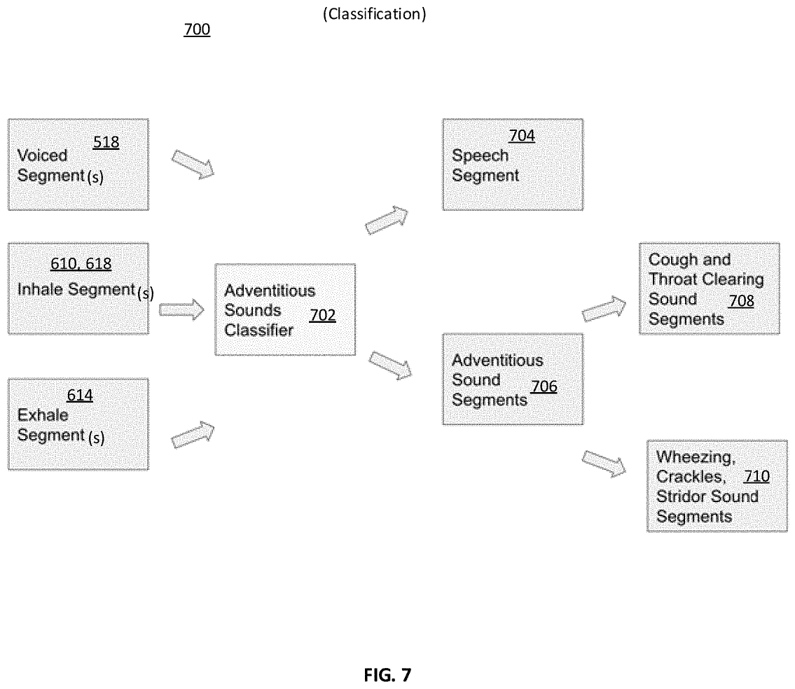

As shown in FIG. 7, the Voice Profiler implements a machine learning classifier model 702 and uses the extracted segments 518, 610, 614, 618 as input into the classifier model 702. The classifier model 702 is trained for classifying sound events, such as coughing, wheezing, stridor, throat clearing and voice crackling. The classifier model 702 classifies the input segments as either a speech segment 704 or an adventitious sound segment 706 based on respiratory events such as coughing, throat clearing, wheezing, crackles, stridor 708, 710. The Voice Profiler extracts qualitative features representing the voice quality portrayed in the classified segments 704, 708, 710.

FIGS. 8A and 8B provide listings 800, 802 of physical state metrics computed from first level features (spectral, temporal) and second level features (metadata, qualitative). As described herein, respective audio portions in the input audio data correspond to vocal actions performed by the speaker in response to instructions provided by one or more prompts. In the listings 800, 802, each metric is associated with a category and calculated with features extracted from segments based on audio performed in response to a recording prompt(s). A calibration prompt to remain silent (“1”) may request a speaker to remain silent for a given amount of time. The silence that occurs in the input audio that corresponds to the speaker's response to the calibration prompt provides audio data from which background noise calibration may be computed. A speech prompt may be a sustained syllable prompt (“2”) to perform a sustained “aah” sound for as long as the speaker is able at a volume of the speaker's normal speaking voice. A non-speech prompt may be a forceful exhale prompt (“3”) that instructs the speaker to inhale deeply, followed by a brief pause and the exhale quickly and forcefully. A speech prompt (“4”) may be a reading passage prompt where a speaker is prompted to read from a predefined reading passage. A speech prompt (“5”) may be a word list passage prompt where a speaker is prompted to read from a list of words that contain a variety of long vowel sounds. Metrics listed in FIGS. 8A and 8B associated with a breathlessness category are metrics calculated based on features extracted during first level segmentation. Metrics that fall in a lung function, adventitious and voice quality category are predictive metrics calculated based on features extracted during second level segmentation. The lung function category metrics include forced vital capacity (“FVC”) (additionally expressed as a fraction of the expected FVC and the expected LLN FVC) and forced expiratory volume (“FEV1”) (additionally expressed as a fraction of the expected FEV1 and the expected LLN FEV1). It is understood that metadata features include attributes such as sex, height, and additional demographic attributes.

The following are non-limiting examples of attributes (i.e., attributes of the speaker) that are predicted from the voice representation in the input audio data/file. Such attributes may correspond to features extracted from segments produced during first and second level segmentation as described herein. Behavioral attributes: dominance, leadership, public and private behavior, and the like. Demographic attributes: race, geographical origins, level of education, and the like. Environmental attributes: location of the speaker at the time of speaking, objects surrounding the person at the time of speaking, devices and communication channels used for voice capture and transmission, and the like. Medical attributes: presence or absence of specific diseases, medication and other substances (e.g., drugs, food, intoxicants, etc.) in the body, state of physical health, state of mental health, effects of trauma, effects of medical procedures, presence or absence of physical abnormalities or disabilities, and the like. Physical attributes: height, weight, body-shape, and facial structure, and the like. Physiological attributes: age, hormone levels, heart rate, blood pressure, and the like. Psychological attributes: personality, emotions, and the like. Sociological attributes: social status, income, profession, and the like.

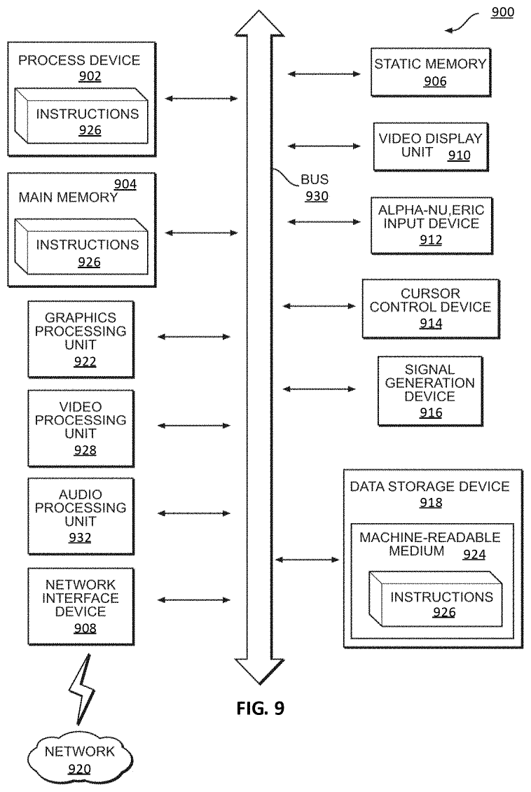

FIG. 9 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 900 includes a processing device 902, a main memory 904 (e.g., read-only memory (ROM), flash memory, dynamic random access memory (DRAM) such as synchronous DRAM (SDRAM) or Rambus DRAM (RDRAM), etc.), a static memory 906 (e.g., flash memory, static random access memory (SRAM), etc.), and a data storage device 918, which communicate with each other via a bus 930.

Processing device 902 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 902 may also be one or more special-purpose processing devices such as an application specific integrated circuit (ASIC), a field programmable gate array (FPGA), a digital signal processor (DSP), network processor, or the like. The processing device 902 is configured to execute instructions 926 for performing the operations and steps discussed herein.

The computer system 900 may further include a network interface device 908 to communicate over the network 920. The computer system 900 also may include a video display unit 910 (e.g., a liquid crystal display (LCD) or a cathode ray tube (CRT)), an alphanumeric input device 912 (e.g., a keyboard), a cursor control device 914 (e.g., a mouse), a graphics processing unit 922, a signal generation device 916 (e.g., a speaker), graphics processing unit 922, video processing unit 928, and audio processing unit 932.

The data storage device 918 may include a machine-readable storage medium 924 (also known as a computer-readable medium) on which is stored one or more sets of instructions or software 926 embodying any one or more of the methodologies or functions described herein. The instructions 926 may also reside, completely or at least partially, within the main memory 904 and/or within the processing device 902 during execution thereof by the computer system 900, the main memory 904 and the processing device 902 also constituting machine-readable storage media.

In one implementation, the instructions 926 include instructions to implement functionality corresponding to the components of a device to perform the disclosure herein. While the machine-readable storage medium 924 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer-implemented method, comprising:
receiving input audio data that includes a representation of a human voice;
extracting one or more voiced segments, one or more non-voiced segments and one or more respiratory event segments from the input audio data; and
predicting a physical state of the speaker of the human voice based on respective attributes of the extracted segments.

2. The computer-implemented method of claim 1, wherein extracting the respective segments comprises:
identifying respective audio portions in the input audio data that correspond to vocal actions performed by the speaker in response to instructions provided by one or more prompts;
wherein each prompt is one of: a prompt to remain silent, one or more types of speech prompts and one or more types of non-speech prompts; and
wherein respective types of respiratory events include respective inhale occurrences and respective exhale occurrences during the vocal actions performed by the speaker.

3. The computer-implemented method of claim 1, wherein extracting the respective segments comprises:
determining a background noise calibration in the input audio data, the input audio data representing a recording of a session during which the speaker responded to one or more prompts;
applying one or more machine learning segmentation models to the input audio data with respect to the background noise calibration; and
receiving, from the one or more machine learning segmentation models, the respective extracted segments isolated from the background noise calibration, the respective extracted segments further comprising: one or more de-noised voiced segments, one or more de-noised respiratory event inhale segments (“de-noised inhale segments”), one or more de-noised respiratory event exhale segments (“de-noised exhale segments”).

4. The computer-implemented method of claim 3, wherein applying one or more machine learning segmentation models to the input audio data with respect to the background noise calibration comprises:
identifying voiced input audio that corresponds to vocal actions performed in response to one or more types of speech prompts;
converting the voiced input audio to a spectrogram representation of the voiced input data;
analyzing one or more regions in the voiced input spectrogram representation according to respective differences in frequency signal intensities indicated in the voiced input spectrogram representation;
detecting at least one region of the voiced input spectrogram representation that exceeds an intensity threshold;
extracting a portion of the voiced input audio that maps to the detected region of the voiced input spectrogram; and
labeling the extracted portion as respective de-noised voiced segment.

5. The computer-implemented method of claim 3, wherein receiving, from the one or more machine learning segmentation models, the respective segments, further comprises:
receiving the one or more de-noised voiced segments, the one or more de-noised forced exhale segments, one or more pause segments and one or more inhale-background segments;
wherein each pause segment is based on audio of respective inhale and exhale occurrences with the background noise; and
wherein each inhale-background segment is based on audio of one or more inhale occurrences with the background noise.

6. The computer-implemented method of claim 5, wherein predicting a physical state of the speaker of the human voice based on respective attributes of the extracted segments comprises:
extracting a first plurality of features from the one or more de-noised voiced segments, the one or more de-noised forced exhale segments, the one or more pause segments and one or more inhale-background segments; and
predicting the physical state of the speaker based at least on the first plurality of features.

7. The computer-implemented method of claim 5, wherein receiving, from the one or more machine learning segmentation models, the respective segments, further comprises:
sending the one or more pause segments, the one or more inhale-background segment and the background noise audio to the one or more machine learning segmentation models; and
receiving, from the one or more machine learning segmentation models, the one or more de-noised inhale segments and the one or more de-noised exhale segments.

8. The computer-implemented method of claim 7, wherein predicting a physical state of the speaker of the human voice based on respective attributes of the extracted segments comprises:
applying a machine learning classifier model to the one or more de-noised voiced segments, the one or more denoised inhale segments, the one or more de-noised exhale segments;
receiving, from the machine learning classifier model, classified segments comprising: one or more speech segments, one or more cough segments and one or more wheezing segments;
extracting a second plurality of features from the respective classified segments; and
predicting the physical state of the speaker based at least on the second plurality of features.

9. A system comprising:
one or more processors; and
a non-transitory computer readable medium storing a plurality of instructions, which when executed, cause the one or more processors to:
receive input audio data that includes a representation of a human voice;
extract one or more voiced segments, one or more non-voiced segments and one or more respiratory event segments from the input audio data; and
predict a physical state of the speaker of the human voice based on respective attributes of the extracted segments.

10. The system of claim 9, wherein extract the respective segments comprises:
identify respective audio portions in the input audio data that correspond to vocal actions performed by the speaker in response to instructions provided by one or more prompts;
wherein each prompt is one of: a prompt to remain silent, one or more types of speech prompts and one or more types of non-speech prompts; and
wherein respective types of respiratory events include respective inhale occurrences and respective exhale occurrences during the vocal actions performed by the speaker.

11. The system of claim 9, wherein extract the respective segments comprises:
determine a background noise calibration in the input audio data, the input audio data representing a recording of a session during which the speaker responded to one or more prompts;
apply one or more machine learning segmentation models to the input audio data with respect to the background noise calibration; and
receive, from the one or more machine learning segmentation models, the respective extracted segments isolated from the background noise calibration, the respective extracted segments further comprising: one or more de-noised voiced segments, one or more de-noised respiratory event inhale segments (“de-noised inhale segments”), one or more de-noised respiratory event exhale segments (“de-noised exhale segments”).

12. The system of claim 11, wherein apply one or more machine learning segmentation models to the input audio data with respect to the background noise calibration comprises:
identify voiced input audio that corresponds to vocal actions performed in response to one or more types of speech prompts;
convert the voiced input audio to a spectrogram representation of the voiced input data;
analyze one or more regions in the voiced input spectrogram representation according to respective differences in frequency signal intensities indicated in the voiced input spectrogram representation;
detect at least one region of the voiced input spectrogram representation that exceeds an intensity threshold;
extract a portion of the voiced input audio that maps to the detected region of the voiced input spectrogram; and
label the extracted portion as respective de-noised voiced segment.

13. The system of claim 11, wherein receive, from the one or more machine learning segmentation models, the respective segments further, comprises:
receive the one or more de-noised voiced segments, the one or more de-noised forced exhale segments, one or more pause segments and one or more inhale-background segments;
wherein each pause segment is based on audio of respective inhale and exhale occurrences with the background noise; and
wherein each inhale-background segment is based on audio of one or more inhale occurrences with the background noise.

14. The system of claim 13, wherein predict a physical state of the speaker of the human voice based on respective attributes of the extracted segments comprises:
extract a first plurality of features from the one or more de-noised voiced segments, the features comprising one or more de-noised forced exhale segments, the one or more pause segments and one or more inhale-background segments; and
predict the physical state of the speaker based at least on the first plurality of features.

15. The system of claim 13, wherein receive, from the one or more machine learning segmentation models, the respective segments further, comprises:
send the one or more pause segments, the one or more inhale-background segment and the background noise audio to the one or more machine learning segmentation models; and
receive, from the one or more machine learning segmentation models, the one or more de-noised inhale segments and the one or more de-noised exhale segments.

16. The system of claim 15, wherein predict a physical state of the speaker of the human voice based on respective attributes of the extracted segments comprises:
apply a machine learning classifier model to the one or more de-noised voiced segments, the one or more denoised inhale segments, the one or more de-noised exhale segments;
receive, from the machine learning classifier model, classified segments comprising: one or more speech segments, one or more cough segments and one or more wheezing segments;
extract a second plurality of features from the respective classified segments; and
predict the physical state of the speaker based at least on the second plurality of features.

17. A computer program product comprising a non-transitory computer-readable medium having a computer-readable program code embodied therein to be executed by one or more processors, the program code including instructions to:
receive input audio data that includes a representation of a human voice;
extract one or more voiced segments, one or more non-voiced segments and one or more respiratory event segments from the input audio data; and
predict a physical state of the speaker of the human voice based on respective attributes of the extracted segments.

18. The computer program product of claim 17, wherein extract the respective segments comprises:
determine a background noise calibration in the input audio data, the input audio data representing a recording of a session during which the speaker responded to one or more prompts;
apply one or more machine learning segmentation models to the input audio data with respect to the background noise calibration; and
receive, from the one or more machine learning segmentation models, the respective extracted segments isolated from the background noise calibration, the respective extracted segments further comprising: one or more de-noised voiced segments, one or more de-noised respiratory event inhale segments (“de-noised inhale segments”), one or more de-noised respiratory event exhale segments (“de-noised exhale segments”).

19. The computer program product of claim 18, wherein apply one or more machine learning segmentation models to the input audio data with respect to the background noise calibration comprises:
identify voiced input audio that corresponds to vocal actions performed in response to one or more types of speech prompts;
convert the voiced input audio to a spectrogram representation of the voiced input data;
analyze one or more regions in the voiced input spectrogram representation according to respective differences in frequency signal intensities indicated in the voiced input spectrogram representation;
detect at least one region of the voiced input spectrogram representation that exceeds an intensity threshold;
extract a portion of the voiced input audio that maps to the detected region of the voiced input spectrogram; and
label the extracted portion as respective de-noised voiced segment.

20. The computer program product of claim 18, wherein receive, from the one or more machine learning segmentation models, the respective segments further comprises:
receive the one or more de-noised voiced segments, the one or more de-noised forced exhale segments, one or more pause segments and one or more inhale-background segments;
wherein each pause segment is based on audio of respective inhale and exhale occurrences with the background noise; and
wherein each inhale-background segment is based on audio of one or more inhale occurrences with the background noise.

21. A system comprising:
one or more processors; and
a non-transitory computer readable medium storing a plurality of instructions, which when executed, cause the one or more processors to:
converting input audio to a spectrogram representation of the input data;
analyzing one or more regions in the spectrogram representation according to respective differences in frequency signal intensities;
detecting at least one region of the spectrogram representation that exceeds an intensity threshold;
extracting a voiced segment from the input audio that maps to the detected region of the input spectrogram; and
predicting a physical state of the speaker of a human voice represented in the input audio based on the extracted voiced segment.
